(19) United States
(12) Patent Application Publication
(10) Pub. No.: US 2011/0263527 A1
(43) Pub. Date: Oct. 27, 2011

(54) Sulfated Galactans With Antithrombotic Activity, Pharmaceutical Composition, Method for Treating or Prophylaxis of Arterial or Venous Thrombosis, Method of Extraction and Use Thereof

(76) Inventors: Paulo Antonio de Souza Mourão, Rio de Janeiro (BR); Fébio Rabelo Melo, Rio de Janeirio-RJ (BR)

(21) Appl. No.: 12/992,411

(22) PCT Filed: May 12, 2009
(86) PCT No.: PCT/BR2009/000125

§ 371 Date: Nov. 12, 2010

(30) Foreign Application Priority Data
May 13, 2008 (BR) PI0801477-9
May 8, 2009 (BR) 020090043919

(51) Int. Cl. A61K 31/737; A61P 7/02; C07H 11/00

(52) U.S. Cl. 514/54; 536/122

(57) ABSTRACT

The present invention relates to low molecular weight sulfated galactans, obtained from algae, particularly genus Botryocladia, preferably species Botryocladia occidentallis, which have no effect on the factor XII activation of the clotting cascade, having antithrombotic heparinoid activity. The present invention also refers to a pharmaceutical composition comprising said sulfated galactans and the use thereof, as heparin substitute, in the treatment or prophylaxis of arterial or venous thrombosis in humans and animals. Furthermore, the present invention provides a method of extraction of the said sulfated galactans.

### FIELD OF INVENTION

The present invention relates to low molecular weight sulfated galactans, preferably obtained from red algae, which have no effect on the factor XII activation of the clotting cascade, giving, however, antithrombotic activity similar to heparin.

The sulfated galactans of the present invention have several advantages before heparins available in the market, particularly low molecular weight heparins, such as: a) they are equally or more active as to low molecular weight heparin in thrombosis models, b) they inhibit thrombosis with low anticoagulant effect, c) they do not cause bleeding, d) they can be obtained from algae rather than from animals, which is the case of heparins available in the market, thus reducing production costs and contamination risks; and e) at low doses, they have a selective action in venous thrombosis models and in high doses, having a role in the arterial thrombosis model.

### BACKGROUND OF THE INVENTION

The main diseases which are fatal to humans involve heart and blood vessels and, consequently, thrombosis (Fareed J, Hoppensteadt D A. Heparins in the new millennium: Will unfractionated heparin survive? Semin Thromb Hemost 2000, 26: 87-88). Once established the diagnosis of venous thromboembolism, there is a need for immediate antithrombotic action to prevent thrombus growth and reduce the risk of pulmonary embolism. Patients with recurrent thromboembolism (e.g. patients with Trousseau's Syndrome) may benefit from administering heparin for long periods and prophylactically. Heparin is also used in the initial treatment of patients with unstable angina or acute myocardial infarction, during and after coronary angioplasty or stenting and during surgery requiring cardiopulmonary bypass. Heparin is also used in the treatment of patients with disseminated intravascular coagulation. Finally, heparin is used as a prophylactic method in invasive procedures such as catheterization.

Heparin has been used for over 50 years and is, currently, the second most used natural drug in the world. The heparin's potent anticoagulant action is achieved mainly through the potentiation of heparin's cofactor II and antithrombin inhibitory actions, the major coagulation enzyme inhibitors, particularly thrombin and factor Xa (Beguin S, Lindhout T, Hemker H C. The effect of trace amounts of tissue factor on thrombin generation in platelet rich plasma, its inhibition by heparin. Thromb Haemostasis 1989; 61: 25-29).

The use of heparin in humans is associated to a number of side effects, including thrombocytopenia, bleeding, inefficiency of action in congenital or acquired deficiencies of antithrombin and inability to inhibit thrombin bound to fibrin. Often, there are abnormalities of liver function tests in patients receiving heparin intravenously or subcutaneously. There are mild rises in liver transaminase activities in plasma, without increased levels of bilirubin or alkaline phosphatase activity. Heparin may also inhibit aldosterone synthesis by suprarenal glands even when low doses are administered.

Heparin is mainly extracted from pig intestinal mucosa or bovine lung, where occurs in low concentration. The incidence of prion-related diseases in mammals and the increasing need for antithrombotic therapy indicate that alternative sources of anticoagulant and antithrombotic compounds are necessary.

Heparin is not the only polysaccharide able to promote thrombin inactivation by antithrombin. A variety of sulfated galactans and fucans from marine algae and invertebrates also promote the same inhibitory effect as disclosed in the following publications: W R L Farias, A P Valente, M S Pereira and P A S Mourão. Structure and anticoagulant activity of sulfated galactans—Isolation of a unique sulfated galactans from the red algae Botryocladia occidentalis and comparison of its anticoagulant action with that of sulfated galactans from invertebrates. J Biol Chem 2000, 275: 29299-29307, Pereira M S, Melo F R, Mourão PAS. Is there a correlation between structure and anticoagulant action of sulfated galactans and sulfated fucans? Glycobiology 2002, 12: 573-580, and Pereira M S, Mulloy B, Mourão PAS. Structure and anticoagulant activity of sulfated fucans—Comparison between the regular, repetitive, and linear fucans from echinoderms with the more heterogeneous and branched polymers from brown algae. J Biol Chem 1999; 274: 7656-7667.

Some patent documents are related to obtaining compositions containing polysaccharides with heparinoid activity, however, neither teaches nor suggests the sulfated galactans of the present invention.

EP 475383 B1 refers to a polysaccharide composition with heparinoid activity obtained from green algae, particularly Codiaceae's family, genus Codium. Also refers to a process for producing said polysaccharide composition as well as an anticoagulant containing, as an active ingredient, said polysaccharide composition. However, the said polysaccharide has not had antithrombotic properties. This is just an anticoagulant agent.

WO 2007/028256 refers to the use of polysaccharide compositions comprising,fucans and galactans to inhibit the release of inflammatory mediators derived from brown and red algae, particularly Ascophyllum nodosum and Asparagopsis armata. It is a mixture of galactans and fucans with anti-inflammatory activity.

Brazilian patent application PI 9808008-3 (corresponding to WO 98/40081) discloses an oligosaccharide composition with anticoagulant and antithrombotic activity obtained from yeast, particularly Pichia holstii and a method of treatment and use of the said composition. Indeed, it is related to oligosaccharides prepared by chemical synthesis (chemical sulfation) without any evidence of antithrombotic activity in vivo.

Brazilian patent applications Nos. PI 9710739-5, PI 9907025-1 and PI 0114007-8 (corresponding to WO 98/03554, WO 99/36443 and WO 02/24754, respectively) describe various synthetic polysaccharides with heparin anticoagulant and antithrombotic activities and pharmaceutical compositions containing the same. These are synthetic polysaccharides with similar properties to heparin, including its side effects such as bleeding.

### The Search for a Heparin's Substitute Antithrombotic

According to S. Alban in his article ‘The precautionary principle’ as a guide for future drug development (European Journal of Clinical Investigation (2005) 35 (Suppl. I), 33-44), heparin is currently the medicinal product derived from pig most widely used in the world and is still the most used drug for prophylaxis and therapy of thromboembolic diseases. In this context, heparin includes non-fractioned heparin and various low molecular weight heparins.

Currently, the viral safety of heparin extracted from pigs is questioned. In line with recent official decisions directed to precaution regarding drugs derived from animals, there are several reasons that make desirable to obtain an alternative to heparins.

After discontinuation of the bovine heparin use as a result of the problem of bovine spongiform encephalopathy or mad cow disease, all heparins used in Europe and North America have been isolated from intestinal mucosa of pigs. But there is a considerable shortage of raw material. According to S. Alban, annually is required mucosa from more than 200 million pigs for heparin production to treat more than 20 million people around the world. In the United States, Germany and France, 312 million doses of heparin were applied in 2003, and the percentage of low molecular weight heparin increased 40%, 79% and 86% respectively. The need is growing, since heparin is used in more countries in a growing number of indications and for longer periods of time. For example, the sales volume of heparin in Germany increased from 45 million Euros in 1986 to 150 million Euros in 2002 (corresponding to 87 million of standard-doses) and sales of heparin doses in the United States increased from 129 million in 2002 to 139 million doses in 2003. Thus, for the production of low molecular weight heparin, more non-fractionated heparin is needed.

Another important aspect is the fact that natural heparin product is a polydisperse mixture of molecules which exhibit wide variations in its composition and its preparations generally contain dermatan sulfate in variable quantities. The said composition of a heparin preparation is dependent on the individual manufacturing processes. Various parameters, ranging from the pig subspecies used, conditions for pig breeding, the process of extraction and final purification of heparin, influence the formation of the final product. As a result, there are large differences between different preparations of heparin, as well as considerable variations in batch, resulting in differences in their biological activities.

Biological activities include not only accelerating the inhibition of thrombin and antithrombin-mediated factor Xa or heparin's cofactor II. Heparin also exhibits a wide range of biological effects, many of them undesirable. Therefore, heparin is not a drug to specific action, but a multivalent biomodulator.

From the development of low molecular weight heparins, some of the disadvantages of non-fractionated heparins have been overcome. The low molecular weight heparins have reduced anticoagulant effect compared with non-fractionated heparin, avoiding excessive bleeding. Furthermore, low molecular weight heparins can be subcutaneously administered while non-fractionated heparins are intravenously administered, which makes its use dependent on constant monitoring. Another advantage of low molecular weight heparins is their long residence in the body compared to non-fractionated heparins.

However, regarding to safety and efficacy, none of these heparins can be considered great.

The study of new polysaccharides with anticoagulant and antithrombotic action is concentrated on two aspects. One involves the search for new compounds with potent and sustained action and without side effects. The other aspect is the use of these compounds as tools to elucidate the molecular and cellular mechanisms involved in thrombosis events.

In this aspect, the present invention comprises sulfated galactans fragments with molecular weight similar to that of low molecular weight heparin. Sulfated galactans fragments of the present invention have high antithrombotic activity and low influence on the change of bleeding time in vivo, thus constituting an important differential before heparin. Although said heparin has antithrombotic activity, it shows increased bleeding time in vivo, which can be considered a limiting to its clinical use.

In some aspects, the ways in which sulfated galactans and heparin assist in antithrombin-mediated thrombin inactivation are quite similar. Both polysaccharides potentiate inactivation of thrombin in similar molar concentrations, interact with the protease in the same region, around exosite II, and promote the formation of a covalently bound complex between thrombin and antithrombin.

However, sulfated galactans fragments and heparin differ in their specific binding site and/or in the antithrombin-induced conformational activation. In tests, sulfated galactans have presented a serpin-independent effect on the coagulation cascade, which has not been verified in heparin. The sulfated polysaccharide has kept its ability in delaying thrombin and factor Xa generation in serpin-free plasma. This is due to inhibition of the protrombinase complex and intrinsic tenase, respectively. The ability of sulfated galactans to inhibit both intrinsic tenase and protrombinase complexes offers an advantage before heparin.

The effect of sulfated galactans on the intrinsic tenase complex was achieved with lower concentrations than that required for prothrombin inhibition suggesting that sulfated galactans are more potent inhibitors of factor Xa generation by the tenase complex than of thrombin formation by the prothrombinase complex.

Despite the effect of heparin on the tenase complex, it did not seem to contribute to the anticoagulant action of this glycosaminoglycan in whole plasma, suggesting that sulfated galactan fragments are more effective than heparin, once that through this mechanism the formation of factor Xa is delayed for more time. In clotting assays and protease generation tests, the presence of serpin was required so that heparin could generate protease related to thrombin and factor Xa formation.

Heparin and sulfated galactan fragment compete with the same biding-site in factor Xa e this binding is crucial for the catalysis of antithrombin-mediated inactivation of the enzyme. (Rezaie, A R. Identification of Basic Residues in the Heparin-binding Exosite of Factor Xa Critical for Heparin and Factor Va Binding. J Biol Chem, 2000; 275: 3320-3327. Assays of protease inactivation by the sulfated polysaccharide in the presence of Ixolaris, a specific ligand of the heparin-binding exosite on factor Xa (Monteiro R Q, Rezaie A R, Ribeiro J M C, Francischetti I M B. Ixolaris: a Factor Xa heparin-binding exosite inhibitor. Biochem J 2005; 387: 871-877)

The observation that the antithrombotic action of heparin may be partly antithrombin-independent, has suggested that the removal of all antithrombin-dependent action of heparin could further reduce its side-effects (such as bleeding risk) and increase the therapeutic action (Kishimoto T K, Viswanathan K, Ganguly T, Elankumaran S, Smith S, Pelzer K, Lansing J C, Sriranganathan N, Zhao G, Galcheva-Gargova Z, Al-Hakim A, Bailey G S, Fraser B, Roy S, Rogers-Cotrone T, Buhse L, Whary M, Fox J, Nasr M, Dal Pan G J et al. Contaminated heparin associated with adverse clinical events and activation of the contact system. N Engl J Med 2008; 358: 2457-67; Holrner, E. in Heparin. Chemical and Biological Properties, Clinical Applications (Lane, D. A, and Lindahl, U., eds). 1989; 575-595; Leizorovicz A, Haugh M C, Chapuis F R, Samama M M, Boissel J P. Low molecular weight heparin in prevention of perioperative thrombosis. BMJ 1992; 305: 913-20; Nurmohamed M T, Rosendaal F R, Büller H R, Dekker E, Hommes D W, Vandenbroucke J P, Briët E. Low-molecular-weight heparin versus standard heparin in general and orthopaedic surgery: a meta-analysis. Lancet 1992; 340: 152-6). Based on this proposition, several researchers attempted to prepare derivatives of heparin with serpin-independent action and, perhaps, antithrombotic effect. One of the approaches involved periodate oxidation of low molecular weight heparin, followed by oversulfation of the saccharide chain which reduces its affinity for antithrombin. This chemically modified heparin became a potent inhibitor of the intrinsic tenase and prothrombinase. complexes (Anderson J A, Fredenburgh J C, Stafford A R, Guo Y S, Hirsh J, Ghazarossian V, Weitz J I. Hypersulfated low molecular weight heparin with reduced affinity for antithrombin acts as an anticoagulant by inhibiting intrinsic tenase and prothrombinase. J Biol Chem 2001; 276: 9755-61). The serpin-independent anticoagulant effect of this modified heparin was predominant in the plasma system. Nevertheless, sulfated galactans from the marine alga B. occidentalis have clear advantage over these previously known anticoagulants in that: 1) it does not require any chemical modification or laborious chemical synthesis; 2) it is not obtained from mammals, reducing the possibility of contamination with prions; and 3) it occurs at high concentrations in an abundant marine plant.

Sulfated galactans have high affinity for thrombin and antithrombin. They interact predominantly with exosite II on thrombin and, similar to heparin, promote the formation of a covalent complex between antithrombin and protease. Antithrobotic effect of sulfated galactans has been tested in experimental models of venous and arterial thrombosis (Farias W R L, Nazareth R A, Mourão P A S. Dual effects of sulfated D-galactans from the red algae Botryocladia occidentalis preventing thrombosis and inducing platelet aggregation, Thromb Haemostasis 2001; 86: 1540-1546; Melo F R, Pereira M S, Monteiro R Q, Foguel D, Mourão P A S. Sulfated galactan is a catalyst of antithrombin-mediated inactivation of α-thrombin. Biochimica et Biophysica Acta 2008; 1780: 1047-1053).

Therefore, the present invention aims to provide sulfated galactans able to act as substitutes or alternative antithrombotics of heparin, featuring improved performance and advantageous features in relation to heparins available in the market, particularly low molecular weight heparins.

Sulfated galactans of the present invention are derived from red algae, particularly of the genus Botryocladia, particularly the species Botryocladia occidentalis and do not have the problems of contamination and safety as mentioned above, characteristic of heparins. Besides the advantage of being obtained from algae and instead of animals, they offer economic advantage. The approximate yield of sulfated galactans of the present invention is about 2% with respect to the initial dry weight of algae from which it was extracted. This high yield means reduction of final cost of the product, compared with heparins available in the market. Moreover, red algae, particularly from the genus Botryocladia, particularly species Botryocladia occidentalis, are easy to cultivate and breed.

Analyzing FIGS. 3A and 3B, it can be noted that costs related to the concentration of used active principle are reduced by at least 75%. According to the graphic, it is only necessary 0.25 mg/kg of sulfated galactans of the present invention to achieve about 80% of antithrombotic activity, whereas to reach approximately 70% of antithrombotic activity is required 1.00 mg/kg of low molecular weight heparin. It can be noted therefore that sulfated galactans of the present invention provide an at least 4 times greater power in relation to low molecular weight heparin.

Therefore, besides economic and health benefits, sulfated galactans of the present invention have the following advantages compared to heparins in the market: a) they are equally or more active on low molecular weight heparin in thrombosis model, b) inhibit thrombosis with low anticoagulant effect, c) do not cause bleeding, d) can be obtained from algae instead of animals, which is the case of heparins available in the market, thus reducing production costs by reducing contamination risks, and e) at low doses, they have a selective action in venous thrombosis models and at high doses, have a preferential role in arterial thrombosis models.

Another advantage to be taken into consideration is that sulfated galactans of the present invention have a body permanence period equivalent to that of low molecular weight heparins.

### Structure of Sulfated Galactans of the Present Invention

Sulfated galactans of the present invention can be obtained from red alga Botryocladia occidentalis and present a repeating structure composed of -4-α-D-Galp-1→3-β-D-Galp-1→. Besides its variable sulfation standard, this molecule presents D-galactose 2,3-di-O-sulfate residues (approximately one third of the total units of α-galactose).

Sulfated galactans of the present invention, which can be obtained from the marine alga Botryocladia occidentalis, has the following repetition structure:

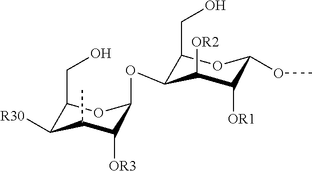

wherein:
R1, R2 e R3=H ou SO₃⁻;
R1 as SO₃⁻≧66%; e
R2 as SO₃⁻≧33%.

This polysaccharide has a repeating structure (-4-α-D-Gal-1→3-β-D-Gal-1→), with a variable pattern of sulfate. Approximately one third of the units is α 2,3-di-sulfated and another one third is 2-sulfate.

The anticoagulant action of sulfated galactans of the present invention, smaller than that observed for low molecular weight heparin, is related to the presence of galactose-2,3-disulfate residues. The more to the right of the graphic, the lower is the anticoagulant activity (FIG. 2). As seen in FIGS. 1B and 2, sulfated galactans of the invention provide a coagulant action significantly lower than the low molecular weight heparin.

When tested in assays with coagulation inhibitors using thrombin as the target protease, native sulfated galactans and non-fractionated heparin have shown similar activities, regardless of whether antithrombin or heparin cofactor II was used as inhibitor (FIGS. 2A and 2C). In contrast, in assays where factor Xa replaces thrombin, the native sulfated galactans require a significantly higher concentration to achieve the same inhibitory effect of non-fractionated heparin (FIG. 2B). Furthermore, it was demonstrated that the formation of thrombin-antithrombin inhibitor complex formed in the presence of native sulfated galactans differs from that formed in the presence of heparin (Melo F R, Pereira M S, Mourão P A S and D Foguel. Antithrombin-mediated anticoagulant activity of sulfated polysaccharides—Different mechanisms for heparin and sulfated galactans. J Biol Chem 2004, 279: 20824-20835).

### OBJECTIVE OF THE INVENTION

Botryocladia native sulfated galactans have antithrombotic effect when tested in experimental thrombosis model. However, the dose-response curve obtained for sulfated galactans is different from that of non-fractionated heparin, since it presents a potent antithrombotic effect at low doses but in high doses this action disappears (FIG. 3A) (Farias W R L, Nazareth R A, Mourão P A S. Dual effects of sulfated D-galactans from the red algae Botryocladia occidentalis preventing thrombosis and inducing platelet aggregation. Thromb Haemostasis 2001, 86: 1540-1546). The high molecular weight native sulfated galactans (≧100 kDa), in contrast to preparations of non-fractionated heparin and low molecular weight heparin (˜14 and ˜5 kDa, respectively) makes difficult the comparison between them. Further, zimogen of factor XII can be activated by contact with negative charges, such as dextran sulfate. Thus, the high content of sulfate, of native sulfated galactans can activate the factor XII, compromising its anticoagulant action. In order to compare the biological action of sulfated galactans with that of non-fractionated heparin and low molecular weight heparin, low molecular weight fragments were prepared from native galactans, with sizes similar to those of non-fractionated heparin and low molecular weight heparin (FIG. 1A). The results indicate that reducing the molecular weight of sulfated galactans minimizes its anticoagulant effect (FIGS. 1B and 2), but also eliminates the unwanted effect on the activation of factor XII (FIG. 4). It should be noticed that the fraction between 2 and 10 kDa, particularly ˜5 kDa, has a low anticoagulant activity (FIGS. 1B and 2). When these fragments were tested in a venous thrombosis model, the fraction of ˜14 kDa showed the same double effect observed for double Sulfated galactans native (FIG. 3B). Surprisingly, the fraction between 2 and 10 kDa, particularly ˜5 kDa, retains the antithrombotic effect in high doses and is capable of preventing thrombosis in both venous and arterial models (FIGS. 3B and D). Considering this, it can be said that the effect of sulfated galactans of the present invention is determined by its dose. Thus, in low doses, its action is selective in venous thrombosis, while in high doses is also shown an effect on arterial thrombosis (FIG. 3B and D). Moreover, it increases the bleeding time (FIG. 6).

Therefore, the main objective of the present invention is to provide sulfated galactans of low molecular weight with antithrombotic effect capable of inhibiting thrombosis with low anticoagulant effect without side effects resulting from the use of heparins available in the market.

The reduction in molecular size has a central role in the dissociation of double effect observed in the pathogenic thrombus formation in vivo. The present invention shows that fragments from 2 to 10 kDa, particularly approximately 5 kDa, of said sulfated galactans are promising drugs for antithrombotic therapy with low or no risk of excessive bleeding.

Furthermore, the present invention provides a method of extraction of sulfated galactans from red alga B. occidentalis.

### BRIEF DESCRIPTION OF THE FIGURES

FIGS. 1A and 1B refer to the size and anticoagulant activity of sulfated polysaccharide. In FIG. 1A, the native sulfated galactans, two fractions of low molecular weight of the said galactans (˜14 kDa (III) and ˜5 kDa (IV)), non-fractionated heparin (UFH) and low molecular weight heparin (LMWH) were separated by PAGE. The molecular weights of low molecular weight sulfated galactans fragments were determined by comparison with the electrophoretic mobility of standard compounds (positions shown at right). In FIG. 1B, citrated human plasma samples were incubated with different concentrations of non-fractionated heparin (□), low molecular weight heparin (▪), native sulfated galactans (▴), ˜14 kDa fragment (◯) and a ˜5 kDa fragment () and used to measure aPTT (activated partial thromboplastin time) [mean±standard error of the mean (SEM), n=3]. For clarity purposes, only one SEM bar is shown for each point.

FIG. 2 shows the dependence of the concentration of sulfated polysaccharides in inactivation of thrombin (A and C) or factor Xa (B) by antithrombin (A and B) or of heparin cofactor II (C). Antithrombin (10 nM) or of heparin cofactor II (15 nM) were incubated with thrombin (2 nM) or factor Xa (2 nM) in the presence of various concentrations of non-fractionated heparin (□), low molecular weight heparin (▪), native sulfated galactans (▴), ˜14 kDa fragment (◯) and a ˜5 kDa fragment (). After 60 seconds, the amidolytic thrombin activity and factor Xa were determined with a chromogenic substrate specific for each protease (A_{450 nm}/min) [mean±standard error of the mean (SEM), n=3]. No inhibition was observed when thrombin or factor Xa was incubated with sulfated polysaccharide alone over the same range of tested concentrations.

FIG. 3 shows the effect of antithrombotic sulfated polysaccharide. Assays were performed in rats with different doses of non-fractionated heparin (□), low molecular weight heparin (▪), native sulfated galactans (▴), ˜14 kDa fragment (◯) and a ˜5 kDa fragment () (mean±standard error of the mean (SEM), n=3). Thrombosis induced model (A and B) in the rats' vena cava. The mean weight of the thrombus for each dose was expressed as a percentage of weight in the absence of polysaccharide, (C) Thrombosis in the arteriovenous shunt model. The mean weight of the thrombus for each dose was expressed as a percentage of the weight of thrombus in the absence of polysaccharide, (D) laser irradiation induced arterial thrombosis model in the carotid artery. Results were expressed as the mean time to complete occlusion of the artery.

FIG. 4 refers to the factor XII activation in human plasma incubated with non-fractionated heparin (□), native sulfated galactans (▴), ˜14 kDa fragment (◯) and a ˜5 kDa fragment (). After 60 seconds of incubation at 37° C., 0.3 mM of chromogenic substrate for plasma calicrein was added. The increase in absorbance at 405 nm was expressed in milli OD/min (mean±standard error of the mean (SEM), n=3).

FIG. 5 shows the anticoagulant activity based on recalcification time. (A) Samples of citrated human plasma were incubated with different concentrations of non-fractionated heparin (□), native sulfated galactans (▴), ˜14 kDa fragment (◯) and a ˜5 kDa fragment () and used to measure the recalcification time (mean±standard error of the mean (SEM), n=3), (B) Samples of rat blood were collected 10 minutes after intravascular administration of different doses of sulfated polysaccharides, the plasma was separated and used to measure the recalcification time ex vivo (mean±standard error of the mean (SEM), n=4). The anticoagulant activity was expressed as T₁/T₀, which is the ratio between the clotting time in the presence or absence of sulfated polysaccharide. The dashed line (T₁/T₀=1) indicates the lack of effect of polysaccharide on coagulation.

FIG. 6 refers to the effect on bleeding. Non-fractionated heparin (□), low molecular weight heparin (▪), native sulfated galactans (▴), and ˜5 kDa fragment () were infused into rats. After 5 minutes, the rats' tails were cut 3 mm from the tip and immersed in 40 mL of distilled water at room temperature. The loss of blood was determined after 60 minutes through the measure of hemoglobin in water. The results were expressed in μL of loss blood (mean±standard error of the mean (SEM), n=4). For clarity purposes only one SEM bar is shown for each point.

### Invention Description

The present invention relates to sulfated galactans with antithrombotic activity, having low molecular weight and comprising a repetition structure (-4-α-D-Galp-1→3-(3-β-D-Galp-1→), wherein total α units comprise α-D-galactopyranose 2,3-di-O-sulfate residues and/or α-D-galactopyranose 2-O-sulfate residues.

Preferably, sulfated galactans of the present invention have molecular weights ranging from 2 to 10 kDa, preferable 5 kDa.

Sulfated galactans of the present invention can be obtained from algae, preferably algae of the genus

Botryocladia, more preferably species Botryocladia occidentalis.

Preferably, total α units of the sulfated galactans of the present invention comprise at least one third of α-D-galactopyranose 2,3-di-O-sulfate residues and at least one third of α-D-galactopyranose 2-O-sulfate residues.

Preferably, repeating structure of sulfated galactans of the present invention comprises the formula:

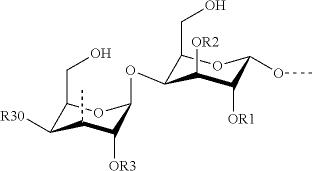

wherein:
R1, R2 e R3=H ou SO₃⁻;
R1 as SO₃⁻≧66%; e
R2 as SO₃⁻≧33%.

Sulfated galactans of the present invention are useful in the treatment of arterial or venous thrombosis in humans or animals.

The present invention also refers to a pharmaceutical composition comprising sulfated galactans of the present invention together With a pharmaceutically accepted vehicle, for example 0.15 M physiologic NaCl solution or any other vehicle known by the one person skilled in the art that is compatible with the pharmaceutical composition of the present invention. This pharmaceutical composition is useful in the treatment of arterial or venous thrombosis in human or animal, particularly venous thrombosis.

In another embodiment the present invention relates to the use of sulfated galactans described above in the treatment or prophylaxis of arterial or venous thrombosis in humans or animals, particularly venous thrombosis.

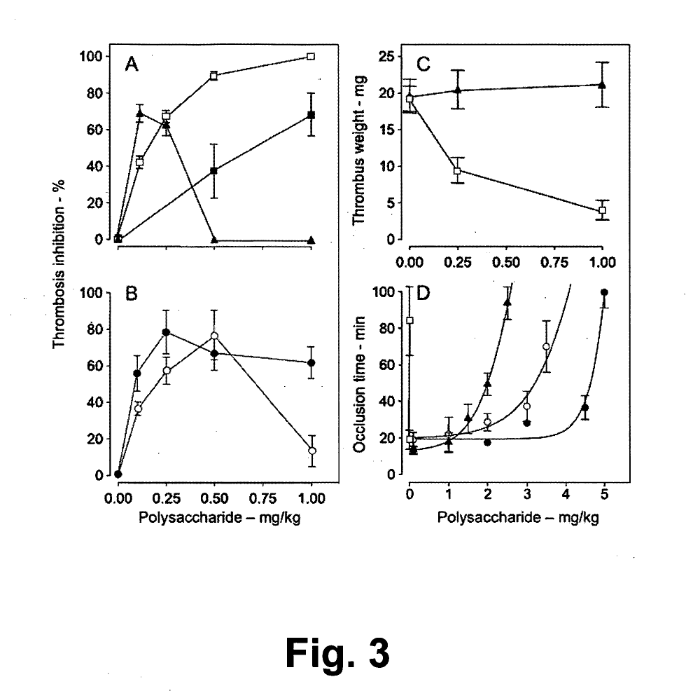

Sulfated galactans of the present invention can act in different concentrations on arterial and venous thrombosis events. Thus, the concentration ranges used in the methods of treatment or prophylaxis of venous thrombosis and arterial thrombosis are different. The range of concentrations of sulfated galactans of the present invention to be administered for the treatment or prophylaxis of venous thrombosis is approximately 0.05 to 5 mg/kg body weight, preferably about 0.25 mg/kg body weight (FIG. 3B). The range of sulfated galactans concentrations of the present invention to be administered for the treatment or prophylaxis of arterial thrombosis is approximately 2 to 10 mg/kg body weight, preferably about 5 mg/kg body weight (FIG. 3D).

The present invention also refers to a method for treatment or prophylaxis of venous thrombosis in humans or animals, comprising administering from 0.05 to 5 mg/kg body weight, preferably 0.25 mg/kg body weight of the sulfated galactans described above to the said human or animal.

Furthermore, the present invention also refers to a method for treatment or prophylaxis of arterial thrombosis in humans or animals, comprising administering from 2 to 10 mg/kg body weight, preferably 5 mg/kg body weight of sulfated galactans described above to the said human being or animal.

It should be understood that the pharmaceutical composition containing said sulfated galactans, for the treatment or prophylaxis of arterial or venous thrombosis, can be administered by any appropriate pathway, e.g. oral, intravenous, subcutaneous, for which it is formulated using the appropriate excipients and pharmaceutically acceptable vehicles for administering thereof.

The present invention is also directed to the use of above-described sulfated galactans in the preparation of a pharmaceutical composition useful in the treatment or prophylaxis of arterial or venous thrombosis in human or animal, particularly venous thrombosis.

In another embodiment the present invention relates to the use of above-described sulfated galactans as a heparin substitute in the treatment or prophylaxis of arterial or venous thrombosis in a human or animal.

In another embodiment the present invention refers to an extraction method of said sulfated galactans.

### DETAILED DESCRIPTION OF THE INVENTION

The study of new polysaccharides with anticoagulant and antithrombotic action is concentrated on two aspects. One involves the search for new compounds with potent and sustained action and without side effects. The other aspect is the use of these compounds as tools to elucidate molecular and cellular mechanisms involved in the thrombosis events. With these objectives, the present invention provides a native sulfated galactans and polysaccharide fragments with molecular weight similar to the non-fractionated heparin and low molecular weight heparin. In particular, the units α-galactose 2,3-di-sulfate are also present in low molecular weight fragments. It is believed that these units constitute structural reasons which confer a high anticoagulant activity of intact sulfated galactans.

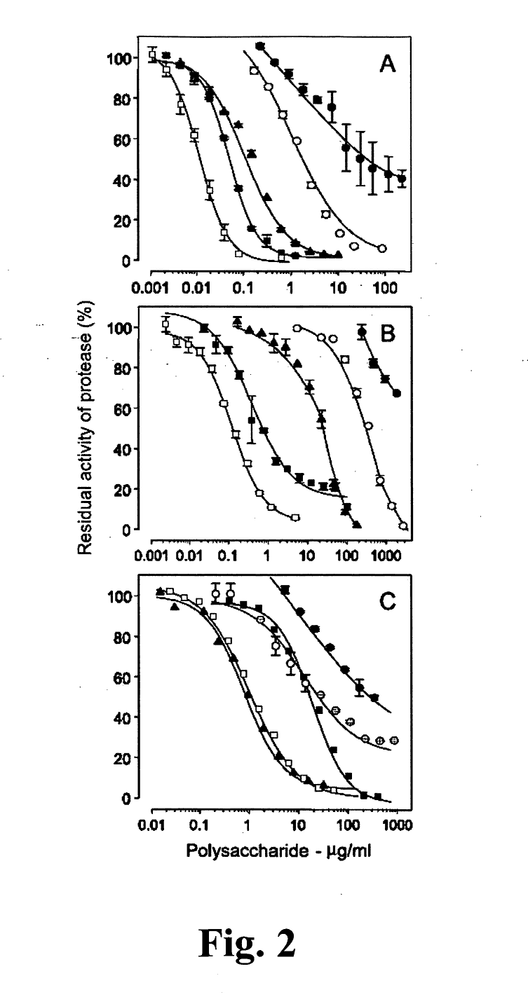

Native sulfated galactans and non-fractionated heparin have similar anticoagulant powers in presence of thrombin (FIGS. 2A and C). The modes by which the non-fractioned heparin and native sulfated galactans help antithrombin-mediated thrombin inactivation are very different. The main difference between the two polysaccharides in this context is that they have different affinities for thrombin (native sulfated galactans>non-fractionated heparin) and antithrombin (non-fractionated heparin>native sulfated galactans). Interestingly, when the data in FIG. 2A are expressed on a molar basis, the inhibitory effects exerted by the two polysaccharides are similar and the thrombin inhibition curve of native sulfated galactans almost overlaps that of non-fractionated heparin. When low molecular weight sulfated galactans fragments were tested, a significant decrease in inhibitory effect on the activity of thrombin and factor Xa exerted by antithrombin or heparin cofactor II (FIG. 2), especially when the results were expressed in a molar basis, was observed. Further, low molecular weight fragments showed a similar anticoagulant activity reduction, independently of the protease or inhibitor used. These results indicate that the reduction in molecular size causes the same effect on different assays.

The arterial thrombosis occurs in regions of moderate shear stress, mainly through the adhesion and aggregation of platelets in the luminal surface of damaged vessels. In contrast, venous thrombosis is mainly related to blood coagulation and resulting deposits of fibrin and red blood cells in stasis or low shear stress regions. The present invention tested the effect of native sulfated galactans and low molecular weight fragments using three experimental thrombosis models. Native sulfated galactans have a potent antithrombotic effect on venous thrombosis in low doses, is less active in arterial model and is inactive in arteriovenous shunt (FIG. 3). In arterial-venous and arterial thrombosis models (FIGS. 3C and D), the antithrombotic effect observed for non-fractionated heparin is clearly associated with its inhibitory action of platelet aggregation. Native sulfated galactans from B. occidentalis has no inhibitory effect on human platelet aggregation and thrombin-induced rats when tested at concentrations up to 0.15 mg/mL and showed some efficacy in inhibition of arterial thrombosis. The sulfated polysaccharides effect on venous thrombosis was based on the assessment of thrombus formation and is mainly associated with its anticoagulant action. The dual effect of native sulfated galactans in the venous thrombosis model was associated with its ability to activate factor XII and this action leads to a different effect on native sulfated galactans on anticoagulant and antithrombotic assays.

The thrombus formation is related to the initiation of the coagulation cascade to finally produce fibrin. The protease factor VIIa present in plasma, together with the membrane protein tissue factor has a central role in this process. Other mechanisms may also contribute to fibrin formation in vivo, as it induces the activation of coagulation factor XII. The old concept that this pathway is irrelevant to coagulation in vivo has recently been changed by the demonstration that mice without factor XII are protected against thromboembolism induced by collagen and epinephrine. The factor XII activation may occur in negatively charged surface of activated platelets. The most important aspect of in vivo observations is the possibility that factor XII may be involved in the thrombus formation (pathological), but not in hemostasis (physiological) since the bleeding time in knockout mice is normal.

Surprisingly, when the molecular weight of sulfated galactans were reduced, the antithrombotic effect in venous model was restored (FIG. 3B). The reduction of fragments' antithrombotic activity in arterial model (FIG. 3D) may be associated with reduced anticoagulant activity (FIG. 2). However, venous antithrombotic action of native sulfated galactans is associated with a balance between anticoagulant activities and pro-coagulant. In low doses, the anticoagulant activity predominates and, in high doses, this action is overcome by activation of factor XII, resulting in a pro-coagulant activity. The decoupling of these actions was observed in experiments with ˜5 kDa fragment, which exhibits a maximum antithrombotic effect at doses similar to those of native sulfated galactans, retaining the action in high doses (FIG. 3B).

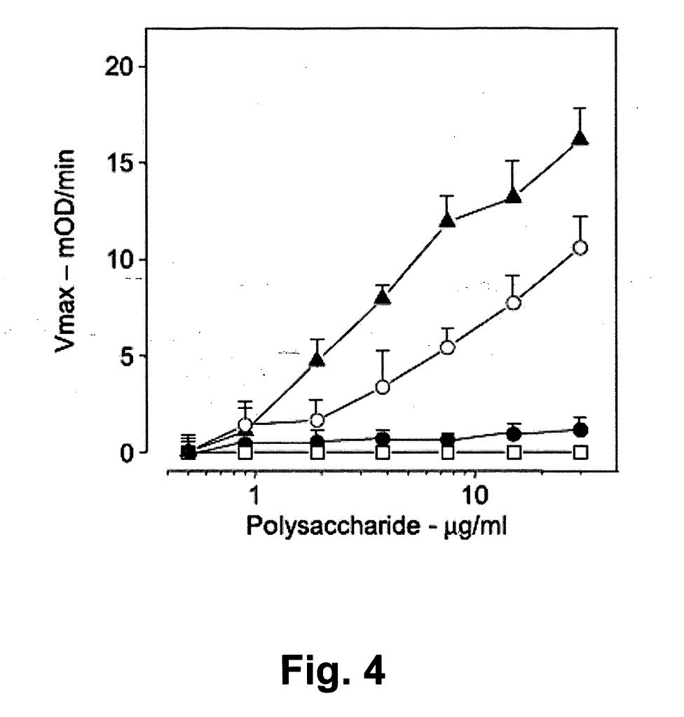

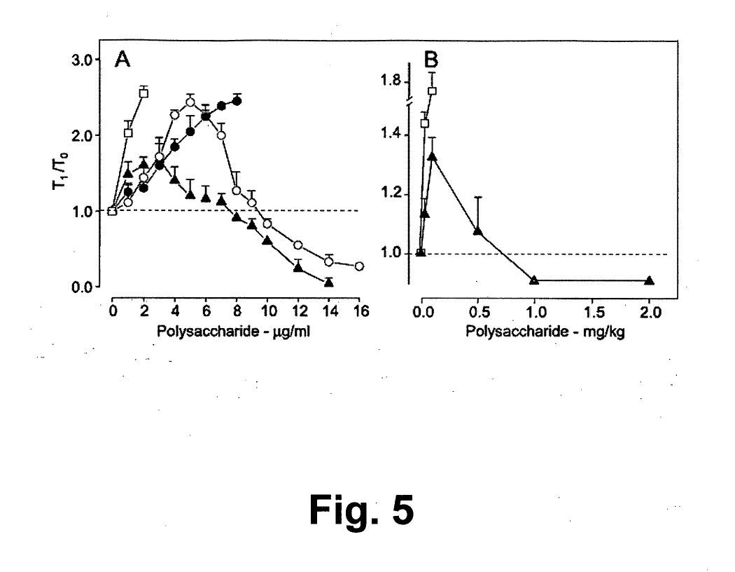

The activation of factor XII in normal plasma involves a slow autodigestion and autoativation of native factor XII linked to a negatively charged surface, followed by a more rapid digestion of factor XII by calicrein. It has been demonstrated that cerebrosides sulfate (sulfatide) provide a very effective surface for reaction during the activation by contact. A similar action was also observed in the presence of dextran sulfate with molecular weight over 8 kDa. Reducing the size of native sulfated galactans also reduces its action on factor XII, so that the ˜5 kDa fragment has not presented this action (FIG. 4). To evaluate the double effect of native sulfated galactans, but also the action of low molecular weight fragments, recalcification tests were carried out in vivo and ex vivo (FIGS. 5A and B). The results were according to those previously observed (FIGS. 3B and 4). It can be obtained an additional demonstration of native sulfated galactans active factor XII using cofactor deficient plasma.

Although sulfated galactans activates factor XII, it does not affect significantly the calicrein-cininogen system, as observed by intravascular administration of polysaccharide, which was not able to change the rat blood pressure.

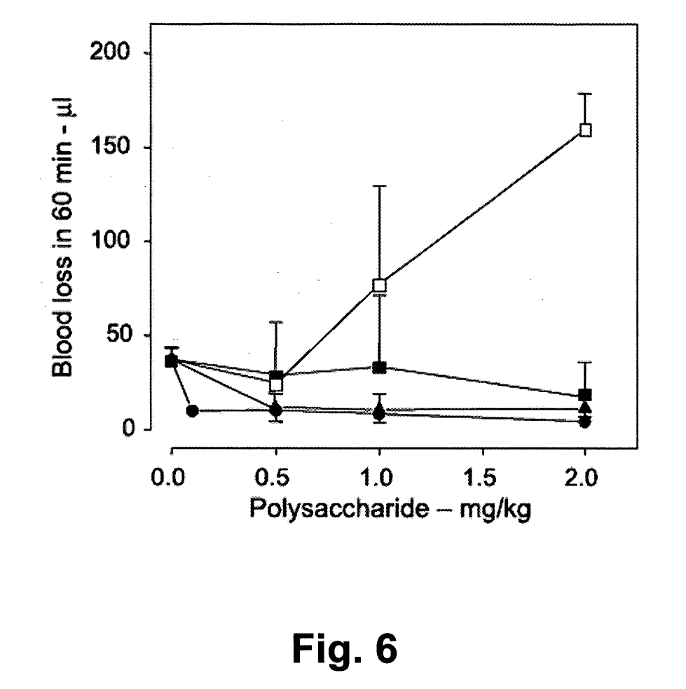

The bleeding effect usually results from modification of blood coagulation induced by the polysaccharide. An increase of aPTT in plasma is generally correlated with an increased bleeding effect. The rat tail bleeding model was used to compare the hemorrhagic effect of polysaccharides. Surprisingly, the native sulfated galactans and its ˜5 kDa fragment had no hemorrhagic effect (FIG. 6). The non-fractionated heparin significantly increased blood loss when injected at 1.0 mg/kg body weight, the same dose in which it has a maximum antithrombotic action in venous model. The Sulfated galactans and its fragments in the range 2 to 10 kDa, particularly ˜5 kDa, did not change the blood loss at the same dose of 2.0 mg/kg body weight, possibly due to the lack of inhibitory effect on platelet aggregation.

Although the native sulfated galactan seems unable to be used to prevent venous thrombosis, the present invention shows that reducing the molecular size has an important role in the dissociation of double effect observed in the pathogenesis of thrombus formation in vivo. Therefore, the present invention provides a low molecular weight fragment, preferably between 2 and 10 kDa, 5 kDa, particularly as a new promising drug for use in antithrombotic therapy with low or no risk of excessive bleeding.

### Experimental Results

### a) Sulfated Polysaccharides

Sulfated galactans were extracted from red algae Botryocladia occidentalis by digestion with protease and purified by ion-exchange chromatography. The purity of each fraction was checked by electrophoresis in agarose gel and NMR spectroscopy, as described in Melo F R, Pereira M S, Mourão P A S and D Foguel. Antithrombin-mediated anticoagulant activity of sulfated polysaccharides—Different mechanisms for heparin and sulfated galactans. J Biol Chem 2004, 279: 20824-20835. The non-fractionated heparin regarding the fourth International Standard (85/502) and low molecular weight heparin (66 IU/mg) of International Standard (85/600) were both obtained from the National Institute for Biological Standards and Control (Potter Bar, UK).

### b) Preparation of Low Molecular Weight Fragments

In an exemplary additional embodiment, the native sulfated galactan, obtained from Botryocladia occidentalis, (40 mg) was dissolved in 0.1 M HCl 1.0 mL and incubated at 60° C. for 60 minutes, then neutralized with 0.1 M NaOH 1.0 ml. Subsequently, a stage of size exclusion chromatography, with a Sephacryl column S-400/HR (220×0.75 cm), balanced with a 0.2 M NH₄HCO₃, pH 7.0, was applied. The column was eluted with the same solution at a flow of 28 mL/h. Fractions of 4 mL were collected and tested by metachromasia using 1,9-dimetilmetilene blue and through the reaction of phenol-H₂SO₄. Various fractions were grouped as four distinct subfractions and then lyophilized.

### c) PAGE

Molecular weights of sulfated polysaccharides were estimated by PAGE. In these experiments, sulfated polysaccharides (˜10 mg each) were applied to a polyacrylamide gel, 1 mm of thickness, in 0.02 M sodium barbital 6%, pH 8.6, and subjected to run for 30 minutes at 100 V. Gels were stained with toluidine blue 0.1% in acetic acid and then washed for ˜4 hours in acetic acid 1%. Molecular weights of low molecular weight sulfated galactans fragments were determined by comparison to electrophoretic mobility of standard compounds.

d) Measurement of Anticoagulant Action of Activated Partial Thromboplastin Time (aPTT)

Coagulation assays were carried out by aPTT according to Anderson's method (Wessler S, Reimer S M, Sheps Me Biologic assay of thrombosis-inducing activity in human serum. J Appl Physiol 1959; 14: 943-946) using citrated human plasma. In these tests, plasma samples (90 μL) were mixed with different amounts of sulfated polysaccharides in 0.15 M NaCl (10μ) and heated for 60 seconds at 37° C., and then 100 μL preheated aPTT reagent (kaolin and a mixture of extracts of phospholipid from rabbit brain and soybean) (Celite-Biolab) were added and incubated for 2 minutes at 37° C. 0.025 M preheated CaCl₂ (100 μL) was then added and aPTT was recorded as required time for formation of a clot in coagulometer (AMELUNG KC4A).

### e) Anticoagulant Activity Based on Recalcification Time

Coagulation tests were performed on plates of 96 wells. First, human citrated plasma samples (90 μL) were mixed with different amounts of sulfated polysaccharides in 0.15 M NaCl (10 μL) and heated for 60 seconds at 37° C. Then, was added 100 μL CaCl₂ 0.25 M and the transmittance at 405 nm was recorded for 720 seconds (plate reader Thermo-max, Molecular Devices). The clot formation time was recorded when the transmittance reached 5%.

### f) Recalcification Time Ex-Vivo

Recalcification assays were performed in rat plasma. Wistar rats (of both sexes, body weight of ˜250 g) were anesthetized with an intramuscular injection of 100 mg/kg body weight of ketamine (Cristália, São Paulo, Brazil) and 16 mg/kg body weight of xylazine (Bayer A S, São Paulo, Brazil). We followed the guidelines for institutional animal care and experimentation. The right carotid artery was cannulated, a solution of sulfated polysaccharides was injected and allowed to circulate for 10 minutes. Blood samples (˜1.0 ml) were collected in sodium citrate 2.8% (9 parts of blood: 1 part citrate). The plasma was separated by centrifugation (1,600×g for 10 minutes), diluted with 3 volumes of TS buffer (Tris/HCl 0.02 M, 0.15 M NaCl, pH 7.4), heated for 60 seconds at 37° C. and then were added 100 μL CaCl₂ 0.2 M. The transmittance at 405 nm was recorded for 720 seconds. The clot formation time was recorded when the transmittance reached 5%.

### g) Thrombin Inhibition or Factor Xa by Antithrombin and Heparin Cofactor II in the Presence of Sulfated Polysaccharides

The incubations were performed on plates of 96 wells. The final reactant concentrations included 10 nM antithrombin or heparin cofactor II 15 nM, thrombin or factor Xa 2 nM (Haematologic Technologies Inc., Vermont, USA) and 0-1,000 g/mL of sulfated polysaccharides in the 40 μL of TS buffer/PEG (Tris/HCl 0.02 M, 0.15 M NaCl and 1.0 mg/mL polyethylene glycol 8000, pH 7.4). Thrombin or factor Xa was added lastly to start reaction. After 60 seconds at 37° C., were added 25 μL chromogenic substrate (S-2238 for thrombin or S-2222 for factor Xa) (Chromogenix AB, Mondal, Sweden), and was recorded absorbance at 405 nm for 120 seconds. The rate of absorbance change was proportional to the activity of thrombin or factor Xa remaining in the incubation. No inhibition occurred in control experiments where thrombin or factor Xa was incubated with antithrombin or heparin cofactor II in the absence of sulfated polysaccharide. Also, no inhibition was observed when thrombin or factor Xa was incubated with sulfated polysaccharide alone over the same range of tested concentrations.

### h) Activation of Factor XII in the Presence of Sulfated Polysaccharides

The assays for activation of factor XII were performed on plates of 96 wells. Citrated human plasma was diluted with 3 volumes of TS buffer and samples (40 μL) were incubated with different concentrations of sulfated polysaccharides (30 μL). After incubation at 37° C. for 60 seconds, were added 30 μL of chromogenic substrate (S-2302, 0.3 mM) (Chromogenix AB, Mondal, Sweden) and absorbance at 405 nm for 300 seconds was recorded. S-2302 is a chromogenic substrate for plasma calicrein activated by its precalicrein precursor through the action of activated factor XII. The method for determining the activity of factor XIIa is based on difference in absorbance between formed p-nitroanilide and original substrate. The formation rate of p-nitroanilide, i.e., the increase in absorbance at 405 nm, is proportional to enzyme activity. No activation occurred in control experiments in the absence of sulfated polysaccharide.

It was verified that dextran sulfate activates factor XII when tested in the above-mentioned test. The effect is associated with the molecular size of the polysaccharide. Only dextran sulfate with molecular weight over 8 kDa activates factor XII.

### i) Assessment of Antithrombotic Properties

### Venous Thrombosis

The antithrombotic activity in rats was investigated using rabbit brain thromboplastin with thrombogenic stimulus. We followed the guidelines for institutional animal care and experimentation. Wistar rats (both sexes, body weight of ˜250 g) were anesthetized with a mixture of ketamine and xylazine, as already described. The inferior vena cava was carefully dissected. A 0.7 cm segment was prepared starting just below the branching of the right renal vein and extending until after the left renal vein which was linked. Different doses of sulfated polysaccharides were intravenously administered 1 cm below the distal lost suture and moved left for 5 minutes. Then, brain thromboplastin (Biolab-Merieux AS, Rio de Janeiro, Brazil) (5 mg/kg body weight) was slowly intravenously injected 1 cm below the loose suture and distal venous segment was closed, first the proximal suture, then the distal suture. After 20 minutes of stasis, the thrombus formed inside the occluded segment was carefully removed, washed with 0.15 M NaCl, dried for 1 hour at 60° C. and weighed.

### Thrombosis in the Arteriovenous Shunt

The antithrombotic activity in the arteriovenous shunt model was investigated according to Umetzu T and Sanai K. Effect of 1-methyl-2-mercapto-5-(3-PYRIDYL)-imidazol (KC-6141), an anti-Aggregating compound on experimental thrombisis in rats, Thromb Haemost, 1979, 39: 74-83. Wistar rats (both sexes, body weight of ˜250 g) were anesthetized with a mixture of ketamine and xylazine, as already described. Through a longitudinal incision in the trachea skin along the midline, the left jugular vein and right carotid artery were linked by an extracorporeal shunt consisting of two 6 cm pieces of polyethylene tube (˜1.3 mm internal diameter). A silk thread with a 5 cm length was stretched in the shunt, leaving a 1 cm outside from the tube. The shunt was filled with a solution of non-fractionated heparin (0.05 mg/kg body weight, which is below the antithrombotic heparin dose). Control animals where the shunt was filled with saline solution showed a thrombus weight similar to control with non-fractionated heparin. After 15 minutes of blood circulation through the shunt (and 20 minutes after intravascular administration of sulfated polysaccharides), blood flow was stopped on the arterial side of the tube and extracorporeal shunt was isolated. From this segment, the silk thread coated with thrombus was carefully pulled by the thread remained outside. The thrombus net weight was immediately determined.

### Arterial Thrombosis

Carotid artery thrombosis was induced using a modification of Eitzman's method (Eitzman D T, Westrick R J, Nabel E G. Plasminogen Activator inhibitor-1 and vitronectin promote vascular thrombosis in mice. Blood 2000, 95: 577-580). Wistar rats (both sexes, body weight of ˜250 g) were anesthetized with a mixture of ketamine and xylazine, as previously described, and tied in the supine position. The right carotid artery was isolated through a cervical incision in the midline and was applied to an ultrasound probe to measure the flow (model 0.5 VB, Transonic Systems, Inc., Ithaca, N.Y., USA). Different concentrations of sulfated polysaccharides were slowly injected in the vena cava. Five minutes after injection of sulfated polysaccharides, rose bengal dye (80 mg/kg body weight, Fisher Scientific Co., Fair Lawn, N.J., USA) was injected and carotid artery was illuminated with a laser beam at 540 nm, 1.5 mW (Melles Griot Inc., Carlsbad, Calif., USA) at a 5 cm distance. The flow in the carotid artery was monitored until complete occlusion occurs.

### j) Effect of Bleeding

Wistar rats (both sexes, body weight ˜250 g) were anesthetized with a mixture of ketamine and xylazine, as previously described. The right carotid artery was cannulated and different polysaccharides doses were infused and allowed to circulate for 5 minutes. The rats tail was cut 3 mm from the tip and immersed in 40 mL of distilled water at room temperature. After 60 minutes, blood loss was determined by measuring the hemoglobin in water by using a spectrophotometric method (Zancan P, Mourão P A S. Venous and arterial thrombosis in rat models: dissociation of the antithrombotic effects of giycosaminoglycans. Blood Coagul Fibrin 2004; 15: 45-54). The blood volume was drawn from a standard curve based on absorbance at 540 nm.

### k) Statistical Analyses

Data expressed as mean±standard error of the mean (SEM) were analyzed by the computer program (Microcal Origin version 3.5).

### Result Analysis

a) Sulfated Galactans Molecular Size versus Anticoagulant Activity Thereof

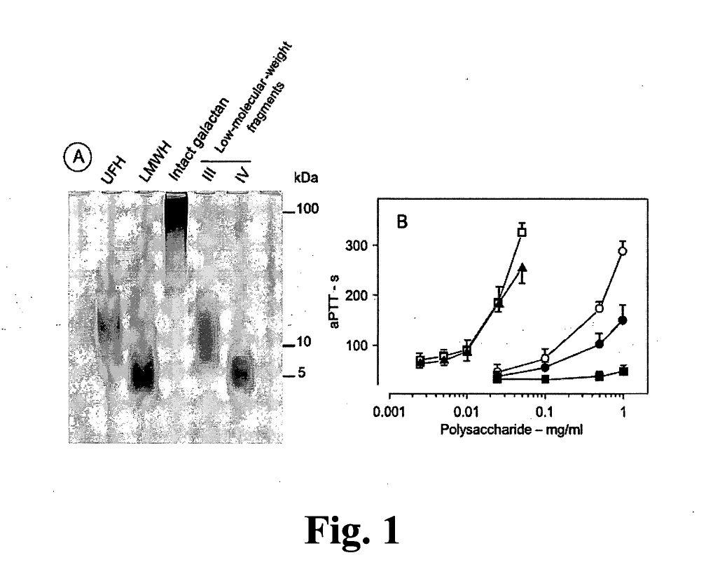

The native sulfated galactan has a molecular weight significantly higher than those of non-fractionated heparin preparations (FIG. 1A), and anticoagulant activity similar when tested by aPTT (FIG. 1B). Both have inhibitory activity when tested in coagulation protease inhibition assays (FIG. 2). Two compounds were quite similar to antithrombin-mediated thrombin inhibition or heparin cofactor II (FIGS. 2A and C), sulfated galactans are not able to achieve the same non-fractionated heparin inhibitory effect for factor Xa inhibition by antithrombin (FIG. 2B) though. However, the obvious difference in molecular size between non-fractionated heparin and native sulfated galactans makes difficult comparison. Thus, native sulfated galactans derivatives with molecular weights close to the non-fractionated heparin and low molecular weight heparin (FIG. 1A) were prepared using mild acid hydrolysis and gel filtration separation. ¹H-NMR spectroscopy showed these derivatives have the same chemical structure and sulfation pattern as native sulfate galactans. Reducing native sulfated galactans molecular weight results in a substantial reduction on anticoagulant activity (FIG. 1B). However, ˜14 and ˜5 kDa fragments also have a qualitatively different anticoagulant action when compared to non-fractionated heparin and low molecular weight heparin, respectively. Non-fractionated heparin is more active than ˜14 kDa fragment in both assays (FIG. 1B and FIG. 2), while low molecular weight heparin is more active than ˜5 kDa fragment only in protease inhibition tests. Then, ˜5 kDa fragment probably interacts in a different manner from that low molecular weight heparin to achieve its inhibitory effect.

### b) Sulfated Galactans Action on Arterial and Venous Thrombosis

Intact sulfated galactans prevent thrombosis in low concentrations in venous model (FIG. 3A), are inactive in arterio-venous shunt (FIG. 3C) and have low activity in arterial model (FIG. 3D). Native sulfated galactans and non-fractionated heparin antithrombotic effects in venous model are probably associated to their anticoagulant actions since venous thrombosis is usually associated to blood coagulation activation and anticoagulant action is predominant in this model. Native sulfated galactans do not inhibit platelet aggregation, a critical stage of arterial thrombosis after endothelial injury, and arterio-venous thrombosis after contact with an irregular surface. However, it is difficult to compare thrombosis models in FIGS. 3A, C and D since non-fractioned heparin and sulfated galactans have very different molecular weights. The strategy was to test the low molecular weight derivatives obtained from native sulfated galactans, close to non-fractionated heparin and low molecular weight heparin. With a reduction in molecular weight, were required higher doses to inhibit arterial thrombosis (FIG. 3D). Surprisingly, with ˜5 kDa fragment, dual effect in venous thrombosis model seen with intact sulfated galactans was lost (FIG. 3B). Anticoagulant and antithrombotic activities decoupling was observed in experiments with ˜5 kDa fragment, which exhibits a maximum antithrombotic effect at doses similar to those of native sulfated galactans, retaining this action in high doses.

c) Sulfated galactans Active Factor XII

In the following experiments, attention was focused on factor XII activation, since factor XII is a coagulation system component which is activated by contact with negative surfaces as dextran sulfate. Obviously, native Sulfated galactans active factor XII, but the reduction in size reduces this unwanted effect (FIG. 4). It should be noticed that ˜5 kDa fragment was not able to activate factor XII even in high doses. However, neither FIG. 4 test nor aPTT test (FIG. 1B) are quite suitable for evaluating the effect of Sulfated galactans in coagulation system. In aPTT tests, high phospholipids concentrations are present and can block sulfated galactans action in factor XII activation. As an alternative, we used a recalcification time test without addition of phospholipids (FIG. 5A). Obviously, non-fractionated heparin has a potent anticoagulant action, while sulfated galactans have a dual effect (up to ˜8 μg/mL, an anticoagulant effect, indicated by T₁/T₀>1; over 8 μg/mL, a procoagulant effect, T₁/T₀<1). ˜14 kDa fragment has a similar effect to native polysaccharide but in a different concentration. These results are in agreement with the proposal that native sulfated galactans have a dual effect on coagulation, inhibiting coagulation proteases at low doses and at high doses prevails the effect on the activation of factor XII.

FIG. 5A experiment does not show a direct relationship between the effect on factor XII in vitro and sulfated galactans effect on venous thrombosis in vivo (FIG. 3A). To join these two effects, sulfated galactans were intravenously injected, as in venous thrombosis assays. The blood was collected and recalcification time was tested ex vivo (FIG. 5B). The dual effect of Sulfated galactans was clear, in contrast with that observed for non-fractionated heparin. The Sulfated galactans showed an anticoagulant effect at the same concentrations observed in venous thrombosis test, while its procoagulant effect occurred at concentrations where antithrombotic effect is absent.

### d) Bleeding Time

In FIG. 6, was measured the sulfated polysaccharides effect on the bleeding time. In this model, non-fractioned heparin significantly increases bleeding time, while low molecular weight heparin, native sulfated galactans and ˜5 kDa fragment had no effect. Particularly, Sulfated galactans of the present invention (˜5 kDa fragment) showed more satisfactory results in relation to low molecular weight heparin. Data also indicate that anticoagulant mechanism and antithrombotic action of hemorrhagic effect of sulfated galactans of the present invention are decoupled.

Furthermore, the present invention provides a method of extraction of sulfated galactans from marine alga B. occidentalis. The said method, which showed to be economically advantageous before already known conventional methods included in the prior art, comprises the following steps:
a) collecting marine algae and dry at 60° C. for 12 hours (16 g).
b) adding 200 mL sodium acetate buffer containing 5 mM EDTA and 5 mM cysteine.
c) adding papain at a ratio of 40% dry weight marine algae.
d) incubating at 60° C. for 12 h.
e) adding to the supernatant, cetyl pyridine chloride (CPC) 10% in proportion of 7.5 mL for each gram of raw material to start.
f) washing the precipitate with distilled water to remove excess of CPC.
g) adding to the precipitate 100 mL of 2 M NaCl/ethanol (100:15, v/v).
h) collecting the supernatant and precipitating sulfated galactans with 2 volumes of absolute ethanol.
i) drying at 60° C. the precipitate containing pure sulfated galactans.
   in which said sulfated galactans obtained in step i) is subsequently hydrolyzed to generate said fragments with antithrombotic activity.

In an additional embodiment of the present invention, said method of extraction can be used for manufacturing a pharmaceutical composition comprising said sulfated galactans for the treatment or prophylaxis of arterial or venous thrombosis in humans or animal.

All documents and publications mentioned in the above specification are incorporated by reference hereby. Various modifications and changes in embodiments of the present invention will be apparent to those skilled in the art without deviating the scope and spirit of the invention. Although the invention has been described with respect to specific embodiments is to be understood that the invention as claimed should not be unduly limited to such specific embodiments. Indeed, various modifications of described embodiments for performing the invention, which are obvious to those skilled in the art, are intended to be within the scope of the claims below.

1. Sulfated galactans with antithrombotic activity having repeating structure according to the following formula: 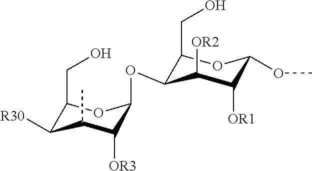
wherein:
R1, R2 and R3 are ═H or SO₃⁻;
R1 is SO₃⁻>66%;, eand
R2 is SO₃⁻>33%.

2. Sulfated galactans according to claim 1, having low molecular weight and comprising repeating structure (-4-α-D-Galp-I→3-β-D-Galp-I→), wherein total α units comprise α-D-galactopyranose 2,3-di-O-sulfate residues and α-D-galactopyranose 2-0-sulfate residues.

3. Sulfated galactans according to claim 1, wherein total α units comprise at least one third of α-D-galactopyranose 2,3-di-O-sulfate residues and at least one third α-D-galactopyranose 2-0-sulfate residues.

4. Sulfated galactans according to claim 1, wherein said low molecular weight is raging from 2 kDa to 10 kD.

5. Sulfated galactans according to claim 4, wherein said molecular weight is 5 kDa.

6. Sulfated galactans according to claim 1, obtained from red algae.

7. Sulfated galactans according to claim 6, wherein the red algae is of the genus Botryocladia.

8. A pharmaceutical composition comprising sulfated galactans as defined in claim 1 and a pharmaceutically acceptable vehicle.

9. A pharmaceutical composition according to claim 8, wherein said composition is used in the treatment or prophylaxis of arterial or venous thrombosis in humans or animals.

10. (canceled)

11. (canceled)

12. (canceled)

13. A method for treating or prophylaxis of venous thrombosis in humans or animals comprising administering from 0.05 mg to 5 mg/kg body weight of said sulfated galactans as defined in claim 1 to the said human or animal.

14. A method for treating or prophylaxis of arterial thrombosis in humans or animals comprising administering from 2 mg to 10 mg/kg body weight of said sulfated galactans as defined in claim 1 to the said humans or animals.

15. A method of extraction of sulfated galactans comprising the following steps:
(a) collecting 16 g of marine algae and drying at a temperature of 60° C. for a period of time of 12 hour;
(b) adding 200 mL of sodium acetate buffer containing 5 mM EDTA and 5 mM cysteine;
(c) adding papain at a ratio of 40% dry weight marine algae;
(d) incubating at a temperature of 60° C. for a period of time of 12 h.
(e) adding to the supernatant, cetyl pyridine chloride (CPC) 10% in proportion of 7.5 mL for each gram of raw material to start;
(f) washing the precipitate with distilled water to remove excess of cetyl pyridine chloride;
(g) adding to the precipitate 100 mL of 2 M NaCl/ethanol (100:15, v/v);
(h) collecting the supernatant and precipitating sulfated galactans with 2 volumes of absolute ethanol; and
(i) drying at a temperature of 60° C. the precipitate containing the pure sulfated galactans in which said sulfated galactans obtained in step (i) is subsequently hydrolyzed to generate said fragments with antithrombotic activity.

16. The method of extraction according to claim 15 for manufacturing a pharmaceutical composition comprising sulfated galactans with antithrombotic activity having repeating structure according to the following formula: 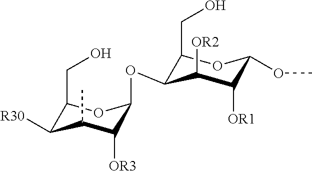
wherein:
R1, R2 and R3 are H or SO₃⁻;
R1 is SO₃⁻>66%, and
R2 is SO₃⁻>33%
and a pharmaceutically acceptable vehicle, for use in the treatment or prophylaxis of arterial or venous thrombosis in humans or animals.

17. Sulfated galactans according to claim 7, wherein the red algae is of the species Botryocladia occidentalis.

18. A method for treating or prophylaxis of venous thrombosis in humans or animals comprising administering 0.25 mg/kg body weight of said sulfated galactans as defined in claim 1, to the said humans or animals.

19. A method, for treating or prophylaxis of arterial thrombosis in humans or animals comprising administering 5 mg/kg body weight of said sulfated galactans as defined in claim 1, to the said humans or animals.
